(19) United States
(12) Patent Application Publication
(10) Pub. No.: US 2012/0157505 A1
(43) Pub. Date: Jun. 21, 2012

(54) ORAL FORMULATIONS OF BENDAMUSTINE

(75) Inventors: Rachel Y. LaBell, Coatesville, PA (US); Piyush R. Patel, Wallingford, PA (US)

(73) Assignee: CEPHALON, INC., Frazer, PA (US)

(21) Appl. No.: 13/284,220

(22) Filed: Oct. 28, 2011

Related U.S. Application Data
(63) Continuation of application No. PCT/US2010/029578, filed on Apr. 1, 2010.
(60) Provisional application No. 61/173,423, filed on Apr. 28, 2009.

(51) Int. Cl. A61K 31/4184; A61P 35/00

(52) U.S. Cl. 514/394

(57) ABSTRACT

The present invention is directed to oral formulations of bendamustine, and its pharmaceutically acceptable salts, methods of use thereof, and methods of treatment comprising them.

### TECHNICAL FIELD

The invention is directed to oral dosage forms of bendamustine, and pharmaceutically acceptable salts thereof.

### BACKGROUND

Bendamustine, 4-{5-[Bis(2-chloroethyl)amino]-1-methyl-2-benzimidazolyl} butyric acid:

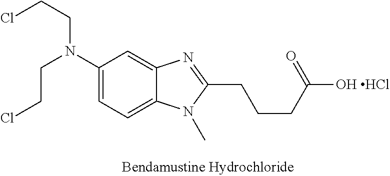

was initially synthesized in 1963 in the German Democratic Republic (GDR) and was available from 1971 to 1992 there under the tradename Cytostasan®. See, e.g., W. Ozegowski and D. Krebs, IMET 3393 γ-[1-methyl-5-bis-(β-chloroethyl)-aminobenzimidazolo-(2)]-butyryl chloride, a new cytostatic agent of the group of benzimidazole nitrogen mustards. Zbl. Pharm. 110, (1971) Heft 10, 1013-1019, describing the synthesis of bendamustine hydrochloride monohydrate. Since that time, it has been marketed in Germany under the tradename Ribomustin®. Bendamustine is an alkylating agent that has been shown to have therapeutic utility in treating diseases such as chronic lymphocytic leukemia, Hodgkin's disease, non-Hodgkin's lymphoma, multiple myeloma, and breast cancer. Currently, bendamustine is available in the United States under the tradename TREANDA® (Cephalon, Inc., West Chester, Pa.). TREANDA® is supplied in single-use vials containing 25 or 100 mg of bendamustine hydrochloride as a lyophilized powder. The lyophilized powder is reconstituted prior to injection.

While bendamustine has been demonstrated as efficacious in its current injectable formulations, it is known that patients receiving such injectable forms of chemotherapeutic treatment would prefer an oral formulation over an injectable one. Oral formulations are generally more convenient and less invasive for the patient, lending to improved patient compliance and outcomes.

It is well known in the art that bendamustine is susceptible to nucleophilic attack by, for example, certain hydroxyl-containing compounds such as water and alkylene glycols such as ethylene glycol and propylene glycol. Many pharmaceutically acceptable excipients include hydroxyl or other nucleophilic groups.

This inherent chemical instability has likely hampered the advancement of oral formulations of bendamustine. Indeed, since its introduction into commercial use over forty years ago, bendamustine has only been provided as injectable formulations, even though published studies have indicated that bendamustine is orally bioavailable. R. Amlacher, et al., Pharmazie, 47 (1992), 378-381; J. Güttner, et al., Arch. Geschwulstforsch. 43/1 (1974), S.; 16-21; A. Härd, et al., Zbl. Pharm. 110 (1971) Heft 10, 1057-1065; U. Horn, et al., Arch. Toxicol., Suppl. 8, 504-506 (1985); R. Preiss, et al., Pharmazie 40 (1985), Heft 11, 782-784; K. Wohlrabe, et al., Zbl. Pharm. 110 (1971) Heft 10, 1045-1047; R. Reszka and P. Scherrer, Offenlegunsschrift DE 103 06 724A1, Sep. 18, 2003. These references suggest that bendamustine might be orally bioavailable. In each study, however, the bendamustine was either dissolved in water just prior to oral ingestion, was provided in neat form in a capsule, or is vesicles.

As such, stable oral dosage forms of bendamustine are needed.

### SUMMARY

The present invention is directed to non-aqueous pharmaceutical compositions for oral administration comprising bendamustine, or a pharmaceutically acceptable salt thereof, and at least one non-aqueous pharmaceutically acceptable excipient selected from the group of solvents and cosolvents such as, for example, propylene carbonate, propylene glycol and polyethylene glycols; surfactants and cosurfactants such as, for example, polysorbates, polyethylene-polypropylene glycol copolymers, and polyethylene glycol stearates, polyethylene glycol laurates; medium chain monoglycerides such as, for example, glyceryl caprylates, caprates and glyceryl monolaurates, polyethylene glycol hydroxy stearates, tocopherol polyethylene glycol 1000 succinate, and triglycerides such as, for example, corn oil. In an embodiment, the present invention is directed to non-aqueous pharmaceutical compositions for oral administration comprising bendamustine, or a pharmaceutically acceptable salt thereof, and at least two non-aqueous pharmaceutically acceptable excipients selected from the group of solvents and cosolvents such as, for example, propylene carbonate, propylene glycol and polyethylene glycols; surfactants and cosurfactants such as, for example, polysorbates, polyethylene-polypropylene glycol copolymers, and polyethylene glycol stearates, polyethylene glycol laurates; medium chain monoglycerides such as, for example, glyceryl caprylates, caprates and glyceryl monolaurates, polyethylene glycol hydroxy stearates, tocopherol polyethylene glycol 1000 succinate, and triglycerides such as, for example, corn oil. Dosage forms comprising the pharmaceutical compositions of the invention are also described, as well as methods of using them.

### DETAILED DESCRIPTION OF ILLUSTRATIVE EMBODIMENTS

As set forth herein, stable, non-aqueous pharmaceutical compositions of bendamustine, or a pharmaceutically acceptable salt thereof, suitable for oral administration, have been prepared. The pharmaceutical compositions of the invention can be a solid solution, solid suspension, solid dispersion, liquid dispersion, suspension, emulsion, microemulsion, gel, or solution and include bendamustine, preferably as its pharmaceutically acceptable salt, for example, bendamustine hydrochloride, and at least one non-aqueous pharmaceutically acceptable excipient. In an embodiment of the present invention, the composition includes at least two non-aqueous pharmaceutically acceptable excipients.

As used herein, “non-aqueous” refers to excipients that do not include water as a major component. Within the scope of the invention, a “major component” will comprise at least 20% (w/w) of the whole. Typically, these non-aqueous excipients will include less than about 2% (w/w) of water. Preferably, these non-aqueous excipients will include less than about 1% (w/w) of water. Anhydrous excipients, i.e., excipient that have no water, are also within the scope of the invention.

The excipients of the invention are chemically stable and nonreactive with bendamustine, or its salts, under one or more of the storage conditions described herein. Excipients suitable for use in the present invention include those identified by the U.S. Food and Drug Administration as Generally Regarded as Safe (GRAS).

Preferred excipients for use in the invention include solvents and co-solvents such as, for example, propylene carbonate, propylene glycol, and polyethylene glycols (for example, PEG 1000 and PEG 1500, PEG 1450, Dow), surfactants and co-surfactants such as, for example, medium chain monoglycerides such as, for example, glyceryl caprylate (for example, CAPMUL MCM, Abitec), glyceryl monolaurates (for example, IMWITOR 312®, Sasol), polyethylene glycol hydroxy stearates (for example, Solutol® HS15, BASF), polysorbates (for example, polysorbate 80), polyethylene-polypropylene glycol copolymers (for example, Poloxamer 188), tocopherol polyethylene glycol 1000 succinate (for example, Speziol® TPGS), triglycerides (for example, corn oil, including super refined corn oil), and polyethylene glycol stearates (for example, Myrj 52), polyethylene glycol laurates, for example polyethylene glycol mono-and dilaurate mixtures (for example Gelucire® 44/14, Gattefossee). See Raymond C. Rowe, et al., Handbook of Pharmaceutical Excipients, APhA Publications, 5^{th} Ed. (2005). Disintegrants, diluents, lubricants, glidants, emulsifying-solubilizing agents, sweetening agents, coating agents, antimicrobial preservatives, and the like, are also within the scope of the invention.

As used herein, “polyethylene glycol,” also known in the art as “PEG,” refers to a polymer of the general formula H(OCH₂CH₂)ₙOH, wherein n is an integer of at least 4. Polyethylene glycols within the scope of the invention include those having a molecular weight of at least 200 g/mol. Preferably, the polyethylene glycols used within the scope of the invention with have molecular weights of from about 400 g/mol to about 8000 g/mol. In preferred embodiments, the polyethylene glycol has a molecular weight of at least about 1000 g/mol. In other embodiments, the polyethylene glycol has a molecular weight of at least about 1500 g/mol.

In certain embodiments, where the excipient selected includes one or more nucleophilic groups, for example, hydroxyl, it is preferred that the nucleophile-containing excipient have a molecular weight of at least 200 g/mol. While not wishing to be bound to any particular theory, it is believed that the larger size inhibits nucleophilic attack of bendamustine by the excipient. Moreover, if the excipient selected includes one or more nucleophilic groups, for example, hydroxyl, it is preferred that the nucleophile-containing excipient comprise less than 40% (w/w) of the composition. Preferably, the nucleophile-containing excipient comprises 20% (w/w) or less of the composition.

Another advantage of the pharmaceutical compositions of the present invention is that they exhibit highly desirable storage stability profiles. Storage conditions can vary and can include variations in temperature, for example, from about 5 ° C. to about 40 ° C., and variations in relative humidity (RH), for example from about 10% RH to about 75% RH. For the purposes of the present application, 5° C. and ambient RH are referred to as “refrigerated conditions,” 25° C. and 60% RH are referred to as “room temperature conditions,” and 40° C. and 75% RH are referred to as “accelerated conditions.” Storage conditions can also include variations in storage time. For example, pharmaceutical compositions of the present invention can be stored for about 1 week, about 1 month, about 2 months, about 3 months, about 6 months, about 1 year, or more. Analysis of the claimed composition can be performed using any technique known in the art, for example, HPLC, GC, and the like.

Preferably, the pharmaceutical compositions of the present invention contain less than about 10% w/w, more preferably, less than about 7% w/w, of degradation impurities after storage of the composition for six months under refrigerated conditions. Preferably, the pharmaceutical compositions of the present invention contain less than about 5% w/w, more preferably, less than about 3% w/w, of degradation impurities after storage of the composition for six months under refrigerated conditions. Preferably, the pharmaceutical compositions of the present invention contain less than about 2% w/w, more preferably, less than about 1% w/w, of degradation impurities after storage of the composition for six months under refrigerated conditions.

Preferably, the pharmaceutical compositions of the present invention contain less than about 10% w/w, more preferably, less than about 7% w/w, of degradation impurities after storage of the composition for six months under room temperature conditions. Preferably, the pharmaceutical compositions of the present invention contain less than about 5% w/w, more preferably, less than about 3% w/w, of degradation impurities after storage of the composition for six months under room temperature conditions. Preferably, the pharmaceutical compositions of the present invention contain less than about 2% w/w, more preferably, less than about 1% w/w, of degradation impurities after storage of the composition for six months under room temperature conditions.

Preferably, the pharmaceutical compositions of the present invention contain less than about 10% w/w, more preferably, less than about 7% w/w, of degradation impurities after storage of the composition for three months under accelerated conditions. Preferably, the pharmaceutical compositions of the present invention contain less than about 5% w/w, more preferably, less than about 3% w/w, of degradation impurities after storage of the composition for three months under accelerated conditions. Preferably, the pharmaceutical compositions of the present invention contain less than about 2% w/w, more preferably, less than about 1% w/w, of degradation impurities after storage of the composition for three months under accelerated conditions. Preferably, the pharmaceutical compositions of the present invention contain less than about 10% w/w, more preferably, less than about 7% w/w, of degradation impurities after storage of the composition for six months under accelerated conditions.

The amount of each excipient used within the scope of the invention will vary, depending on the particular excipients selected. Preferably, the pharmaceutical compositions of the invention will include at least one, and in another embodiment, two non-aqueous pharmaceutically acceptable excipients. In the case of two such excipients, the ratio of each excipient will be from about 1:1 to about 1:4. Ratios of about 3:7 may also be preferred.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

In some embodiments, the pharmaceutical compositions can be prepared in accordance with acceptable pharmaceutical procedures, such as described in Remington's Pharmaceutical Sciences, 17th edition, ed. Alfonoso R. Gennaro, Mack Publishing Company, Easton, Pa. (1985).

The dosage forms of the invention are intended to be administered orally. As such, the dosage forms of the invention may also comprise solid carriers known in the art. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient, i.e. bendamustine or a pharmaceutically acceptable salt thereof. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Suitable drug dosage forms include, but are not limited to, tablets, for example, immediate-, controlled-, and extended-release tablets, pills, capsules, soft gels, sachets, granules, powders, chewing gums, suspensions, emulsions, and solutions. Particularly preferred are tablets and capsules of all varieties. Where appropriate and necessary, the pharmaceutical compositions and dosage forms of the invention may include diluents, binding agents, dispersing agents, surface-active agents, lubricating agents, coating materials, flavoring agents, coloring agents, controlled release formulations, sweeteners or any other pharmaceutically acceptable additives, for example, gelatin, sodium starch glycolate, lactose, starch, talc, magnesium stearate, microcrystalline cellulose, Povidone, hydrogenated or unsaturated oils, polyglycols, syrups or other aqueous solutions. Where the formulations are tablets or capsules and the like the formulations may be presented as premeasured unit doses or in multidose containers from which the appropriate unit dose may be withdrawn.

Liquid carriers may be used in preparing solutions, suspensions, emulsions, syrups, and elixirs. The active ingredient of this invention, i.e. bendamustine or a pharmaceutically acceptable salt thereof, can be dissolved or suspended in a pharmaceutically acceptable liquid carrier such as an organic solvent or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral administration alcohols (including monohydric alcohols and polyhydric alcohols e.g. glycols) and their derivatives, oils (e.g. fractionated coconut oil and arachis oil), and for short contact periods, water (particularly containing additives as above, e.g. cellulose derivatives, preferably sodium carboxymethyl cellulose solution).

Preferably the pharmaceutical composition is in single unit dosage form, e.g. as tablets, capsules, powders, solutions, suspensions, emulsions, or granules. In such form, the composition is sub-divided in unit dose containing appropriate quantities of the active ingredient; the unit dosage forms can be packaged compositions, for example packeted powders, vials, ampoules, and the like. The unit dosage form can be, for example, a capsule or tablet itself, or it can be the appropriate number of any such compositions in package form.

The following examples are offered for illustrative purposes, and are not intended to limit the invention in any manner. Those of skill in the art will readily recognize a variety of noncritical parameters which can be changed or modified to yield essentially the same results.

### EXAMPLES

Materials

The materials used in the Examples were obtained from the sources described in Table 1. Bendamustine HCl (BMl) was prepared according to known methods.

TABLE 1

| Material | Source | Material | Source |
|---|---|---|---|
| Capmul ® MCM | Abitec | PEG1500 | Dow Chemical |
| | Corporation | | Company |
| Imwitor ® 312 | Sasol North | Polysorbate 80 | Spectrum |
| | America | | Chemical, |
| | | | Mfg. Corp. |
| Myrj ® 52 | Croda Inc. | Propylene | Arcos |
| | | Carbonate (“PC”) | |
| PEG1000 | Dow Chemical | Propylene Glycol | EMD Bio- |
| | Company | (“PG”) | sciences, Inc. |
| Super Refined ® | Croda Inc. | Poloxomer 188 | BASF |
| Corn Oil | | | |
| Gelucire 44/14 | Gattefossee | Solutol HS 15 | BASF |
| Speziol TPGS | Cognis | PEG 4500 | Dow Chemical |
| | | | Co. |

Where mixtures of excipients were used, the excipients were pre-mixed before being added to bendamustine hydrochloride. Unless otherwise indicated, all compositions are percentage based on weight.

Analytical Methods

HPLC Method for Example 1

Samples were analyzed by injecting 2 μL of test material into a Zorbax Bonus-RP

Column (150×4.6 mm, 5 μm packing) set at 30° C., with a total flow rate of 1.0 mL/min. The column employed a VWD detector set at 254 nm. The mobile phase consisted of a two-phase gradient flow, with the first mobile phase A consisting of 0.1% TFA in water (v/v) and the second mobile phase B consisting of 0.1% TFA in acetonitrile (v/v), according to the gradient shown in Table 2.

TABLE 2
Flow gradients for HPLC method

| | | | Flow |
|---|---|---|---|
| Time (min) | A [symbol] (%) | B (%) | (mL/min) |
| 0 | 9 [symbol] 3 | 7 | 1.0 |
| 5.0 | 9 [symbol] 3 | 7 | 1.0 |
| 13.0 | 7 [symbol] 3 | 27 | 1.0 |
| 16.0 | 7 [symbol] 3 | 27 | 1.0 |
| 25.0 | 4 [symbol] 3 | 57 | 1.0 |
| 26.0 | 1 [symbol] 0 | 90 | 1.0 |
| 31.0 | 1 [symbol] 0 | 90 | 1.0 |

[symbol]

indicates data missing or illegible when filed

HPLC Method for Example 2

Samples were analyzed by injecting 5 μL of test material into a Zorbax Bonus-RP Column (150×4.6 mm, 3.5 μm packing) set at 30° C., with a total flow rate of 1.5 mL/min. The column employed a VWD detector set at 254 nm. The mobile phase consisted of a two-phase gradient flow, with the first mobile phase A consisting of 0.1% TFA in water (v/v) and the second mobile phase B consisting of 0.1% TFA in acetonitrile (v/v), according to the gradient shown in Table 2A.

TABLE 2A
Gradient:

| Time (min.) | % A | % B |
|---|---|---|
| 0.0 | 93 | 7 |
| 3.3 | 93 | 7 |
| 6.7 | 73 | 27 |
| 10.7 | 73 | 27 |
| 16.0 | 43 | 57 |
| 16.1 | 10 | 90 |
| 18.0 | 10 | 90 |
| 18.1 | 93 | 7 |
| 20.0 | 93 | 7 |

### Example 1

Preparation of Formulations for Stability Testing of Bendamustine HCl

Using Two and Three Excipients

Excipient formulations were prepared (%w/w, see Table 3A for excipient combinations and ratios) and bendamustine HCl (50 mg/mL) was added. The solids were heated to 60° C. and stirred for 2 hours and then cooled to room temperature and allowed to stand overnight. Solid samples were then melted and 300 μL from each vial was pipetted into clean vials using a positive displacement pipette. Liquid formulations were mixed at room temperature for 3 hrs and sampled in the same manner as the solids. The individual vials were placed on stability testing at 40° C/75% RH, 30° C/65% RH, 25° C/60% RH, and 5° C. Samples were prepared by a 25-times dilution in methanol, and analyzed according to the HPLC method described above for Example 1. The results are reported in Tables 3A through 3E.

As used herein, “% purity bendamustine” refers to the area under the curve of the peak corresponding to bendamustine of a sample pharmaceutical composition and is exclusive of non-bendamustine peaks. BMl is bendamustine HCl. Table 3A shows initial purity for each of the given compositions.

TABLE 3A

| | % Purity |
|---|---|
| Formulation | bendamustine |
| 5:4:1 PC:PG:Polysorbate 80 | 99.49 |
| 8:2 Corn oil:Polysorbate 80 | 99.62 |
| 4:1 Imwitor 312:PG | 99.23 |
| 1:1 Imwitor 312:Poloxamer 188 | 99.49 |
| 2:1 Capmul MCM:Poloxamer 188 | 99.37 |
| 1:1 PEG1000:Myrj 52 | 99.06 |
| 4:1 Imwitor:Polysorbate 80 | 98.85 |
| 1:4 PG:Poloxamer 188 | 99.27 |
| 7:3 PEG1500:Poloxamer 188 | 99.46 |
| 1:1 PG:Polysorbate 80 | 99.43 |

TABLE 3B
Purity of BM1 at 40° C./75% RH

| | | 1 | 2 | 3 | 4 | 6 |
|---|---|---|---|---|---|---|
| Formulation | Initial | week | weeks | weeks | weeks | weeks |
| 5:4:1 PC:PG:Poly- | 99.49 | 96.20 | 89.14 | NT | NT | NT |
| sorbate 80 | | | | | | |
| 8:2 Corn oil:Poly- | 99.62 | 98.87 | 94.26 | 73.88 | NT | NT |
| sorbate 80 | | | | | | |
| 4:1 Imwitor 312:PG | 99.23 | 93.48 | 61.41 | NT | NT | NT |
| 1:1 Imwitor 312: | 99.49 | 99.45 | 99.17 | 99.13 | 99.34 | 99.28 |
| Poloxamer 188 | | | | | | |
| 2:1 Capmul MCM: | 99.37 | 98.97 | 98.92 | 98.56 | 98.52 | 98.92 |
| Poloxamer 188 | | | | | | |
| 1:1 PEG1000:Myrj | 99.06 | 99.07 | 97.17 | 98.71 | 98.80 | 97.74 |
| 52 | | | | | | |
| 4:1 Imwitor:Poly- | 98.85 | 98.29 | 97.50 | 97.02 | 97.10 | 98.44 |
| sorbate 80 | | | | | | |
| 1:4 PG:Poloxamer | 99.27 | 98.82 | 98.38 | 97.66 | 97.52 | 97.18 |
| 188 | | | | | | |
| 7:3 PEG1500: | 99.46 | 99.43 | 99.10 | 99.18 | 99.32 | 99.44 |
| Poloxamer 188 | | | | | | |
| 1:1 PG:Polysorbate | 99.43 | 95.46 | 92.45 | 81.09 | NT | NT |
| 80 | | | | | | |

NT = not tested

TABLE 3C
Purity of BM1 at 30° C./65% RH

| | | 4 | 6 | 8 | 13 | 18 |
|---|---|---|---|---|---|---|
| Formulation | Initial | weeks | weeks | weeks | weeks | weeks |
| 5:4:1 PC:PG:Poly- | 99.49 | 93.62 | 95.47 | 47.95 | NT | NT |
| sorbate 80 | | | | | | |
| 8:2 Corn oil:Poly- | 99.62 | 99.09 | 99.09 | 84.22 | NT | NT |
| sorbate 80 | | | | | | |
| 4:1 Imwitor 312:PG | 99.23 | 91.81 | 89.40 | NT | NT | NT |
| 1:1 Imwitor 312: | 99.49 | NT | NT | 99.12 | 98.95 | 98.71 |
| Poloxamer 188 | | | | | | |
| 2:1 Capmul MCM: | 99.37 | NT | NT | 98.73 | 97.90 | 96.01 |
| Poloxamer 188 | | | | | | |
| 1:1 PEG1000:Myrj | 99.06 | NT | NT | 98.04 | 97.06 | 97.14 |
| 52 | | | | | | |
| 4:1 Imwitor:Poly- | 98.85 | NT | NT | 98.35 | 97.86 | 97.81 |
| sorbate 80 | | | | | | |
| 1:4 PG:Poloxamer | 99.27 | NT | NT | 96.91 | 95.08 | 94.56 |
| 188 | | | | | | |
| 7:3 PEG1500: | 99.46 | NT | NT | 99.43 | 99.40 | 99.39 |
| Poloxamer 188 | | | | | | |
| 1:1 PG:Polysorbate | 99.43 | 92.47 | NT | NT | NT | NT |
| 80 | | | | | | |

NT = not tested

TABLE 3D
Purity of BM1 at 25° C./60% RH

| | | 4 | 8, 9 | 12, 13 | 17 |
|---|---|---|---|---|---|
| Formulation | Initial | weeks | weeks | weeks | weeks |
| 5:4:1 PC:PG:Polysorbate 80 | 99.49 | 97.74 | 94.06 | 40.72 | NT |
| 8:2 Corn oil:Polysorbate 80 | 99.62 | 99.32 | 79.99 | NT | NT |
| 4:1 Imwitor 312:PG | 99.23 | 94.52 | 82.50 | NT | NT |
| 1:1 Imwitor | 99.49 | 99.41 | NT | NT | NT |
| 312:Poloxamer 188 | | | | | |
| 2:1 Capmul | 99.37 | 99.20 | NT | NT | NT |
| MCM:Poloxamer 188 | | | | | |
| 1:1 PEG1000:Myrj 52 | 99.06 | 98.61 | NT | NT | NT |
| 4:1 Imwitor:Poly- | 98.85 | 98.57 | NT | NT | NT |
| sorbate 80 | | | | | |
| 1:4 PG:Poloxamer 188 | 99.27 | 98.50 | NT | NT | NT |
| 7:3 PEG1500:Poloxamer | 99.46 | 99.43 | NT | NT | NT |
| 188 | | | | | |
| 1:1 PG:Polysorbate 80 | 99.43 | NT | NT | 94.08 | 26.12 |

NT = not tested

TABLE 3E
Purity of BM1 at 5° C.

| Formulation | Initial | 9 weeks | 13 weeks | 18 weeks |
|---|---|---|---|---|
| 5:4:1 PC:PG:Polysorbate 80 | 99.49 | NT | 99.20 | 99.04 |
| 8:2 Corn oil:Polysorbate 80 | 99.62 | NT | 99.44 | 99.43 |
| 4:1 Imwitor 312:PG | 99.23 | 97.98 | 98.52 | 97.88 |

NT = not tested

### Example 2

Preparation of Formulations for Stability Testing of Bendamustine HCl Using One Excipient

250 mg of bendamustine hydrochloride was weighed into a glass vial and 5 g of melted excipient were added. This was stirred at 70° C. for three hours and then cooled to room temperature and allowed to stand overnight. Solid samples were then melted and 350 μL from each vial was pipetted into clean vials using a positive displacement pipette. Liquid formulations were mixed at room temperature for 3 hrs and sampled in the same manner as the solids. The individual vials were placed on stability testing at 40° C/75% RH, 30° C/65% RH, and 25° C/60% RH. Samples were prepared by a 25-times dilution in methanol, and analyzed according to the HPLC method described above for Example 2. Stability results are provided in Tables 4A, 4B and 4C.

TABLE 4A
Purity of BM1 at 25° C./60% RH
% BM1
Purity

| Excipient | Initial | 3 Mo | 6 Mo |
|---|---|---|---|
| Myrj 52 | 98.6 | 96.8 | 96.2 |
| Poloxamer 188 | 99.7 | 99.6 | 99.6 |
| Speziol TPGS | 99.7 | 99.6 | 99.7 |
| PEG 1450 | 99.3 | 99.3 | 99.1 |
| Gelucire 44/14 | 99.6 | 99.5 | 99.6 |
| Imwitor 312 | 99.4 | 95.3 | 91.3 |
| Solutol HS15 | 98.3 | 96.7 | 95.1 |

TABLE 4B
Purity of BM1 at 30° C./65% RH

| | % BM1 Purity | | | | | | |
|---|---|---|---|---|---|---|---|
| Excipient | Initial | 1 Mo | 2 Mo | 3 Mo | 4 Mo | 5 Mo | 6 Mo |
| Myrj 52 | 98.6 | 97.1 | 96.4 | 96.3 | 95.7 | 96.1 | 95.6 |
| Poloxamer 188 | 99.7 | 99.7 | 99.7 | 99.7 | 99.7 | 99.4 | 99.6 |
| Speziol TPGS | 99.7 | 99.6 | 99.6 | 99.6 | 99.7 | 99.6 | 99.6 |
| PEG 1450 | 99.3 | 99.1 | 98.9 | NT | 99.1 | 95.6 | 98.7 |
| Gelucire 44/14 | 99.6 | 99.6 | 99.6 | 99.5 | 99.3 | 99.5 | 99.4 |
| Imwitor 312 | 99.4 | 96.8 | 93.2 | 90.2 | 87.5 | NT | NT |
| Solutol HS15 | 98.3 | 96.0 | 95.4 | 95.9 | 95.4 | 99.6 | 95.3 |

TABLE 4C
Purity of BM1 at 40° C./75% RH

| | % BM1 Purity | | | | | | |
|---|---|---|---|---|---|---|---|
| Excipient | Initial | 1 Mo | 2 Mo | 3 Mo | 4 Mo | 5 Mo | 6 Mo |
| Myrj 52 | 98.6 | 96.7 | 95.8 | 95.6 | 95.4 | 95.8 | 95.6 |
| Poloxamer 188 | 99.7 | 99.6 | 99.7 | 99.6 | 99.5 | 99.4 | 99.5 |
| Speziol TPGS | 99.7 | 99.7 | 99.6 | 99.6 | 99.6 | 99.1 | 99.6 |
| PEG 1450 | 99.3 | 90.8 | 87.8 | NT | NT | NT | NT |
| Gelucire 44/14 | 99.6 | 99.2 | 99.2 | 60.5 | NT | NT | NT |
| Imwitor 312 | 99.4 | 72.8 | 31.5 | NT | NT | NT | NT |
| Solutol HS15 | 98.3 | 94.4 | 92.2 | 88.5 | NT | NT | NT |

Thus, in a first aspect of the present invention, there is provided a non-aqueous pharmaceutical composition for oral administration comprising:

bendamustine, or a pharmaceutically acceptable salt thereof; and

at least one non-aqueous pharmaceutically acceptable excipient selected from the group consisting of solvents and co-solvents, surfactants and co-surfactants, medium chain monoglycerides, and triglycerides.

A second aspect of the present invention provides a non-aqueous pharmaceutical composition for oral administration comprising:

bendamustine, or a pharmaceutically acceptable salt thereof; and

at least two non-aqueous pharmaceutically acceptable excipients selected from the group consisting of solvents and co-solvents, surfactants and co-surfactants, medium chain monoglycerides, and triglycerides.

A third aspect of the present invention provides the non-aqueous pharmaceutical composition of the first aspect, wherein the at least one non-aqueous pharmaceutically acceptable excipient is selected from the group consisting of propylene carbonate, propylene glycol, glyceryl caprylate, polysorbates, polyethylene-polypropylene glycols, corn oil, glyceryl monolaurates, polyethylene glycol monostearates, polyethylene glycol monolaurates, polyethylene glycol dilaurates, polyethylene glycol hydroxyl stearates, triglycerides, polyethylene glycol distearates, polyethylene glycol tocopherols, and polyethylene glycols.

A fourth aspect provides the non-aqueous pharmaceutical composition of the second aspect, wherein the at least one non-aqueous pharmaceutically acceptable excipient is selected from the group consisting of propylene carbonate, propylene glycol, glyceryl caprylate, polysorbates, polyethylene-polypropylene glycols, corn oil, glyceryl monolaurates, polyethylene glycol monostearates, polyethylene glycol monolaurates, polyethylene glycol dilaurates, polyethylene glycol hydroxyl stearates, triglycerides, polyethylene glycol distearates, polyethylene glycol tocopherols, and polyethylene glycols.

A fifth aspect provides a non-aqueous pharmaceutical composition for oral administration comprising:

bendamustine, or a pharmaceutically acceptable salt thereof; and

at least one non-aqueous pharmaceutically acceptable excipient selected from the group consisting of a polyethylene glycol monostearate, a polyethylene-polypropylene glycol, tocopherol polyethylene glycol 1000 succinate, a polyethylene glycol, a polyethylene glycol mono- and dilaurate mixture, a glyceryl laurate, and a polyethylene glycol hydroxystearate mixture.

A sixth aspect provides a non-aqueous pharmaceutical composition for oral administration according to the fifth aspect comprising:

bendamustine, or a pharmaceutically acceptable salt thereof; and at least one non-aqueous pharmaceutically acceptable excipient selected from the group consisting of Myrj 52, Poloxamer 188, Speziol TPGS, PEG 1450, Gelucire 44/14, Imwitor 312, and Solutol HS15.

A seventh aspect provides the non-aqueous pharmaceutical composition of the second or fourth aspect, wherein the pharmaceutical composition is a solid solution, solid suspension, solid dispersion, liquid dispersion, suspension, emulsion, microemulsion, gel, or solution.

An eighth aspect provides the non-aqueous pharmaceutical composition of the first or third aspect, wherein the pharmaceutical composition is a solid solution, solid suspension, solid dispersion, liquid dispersion, suspension, gel, or solution.

A ninth aspect provides the non-aqueous pharmaceutical composition of the fourth aspect, wherein the at least two non-aqueous pharmaceutically acceptable excipients are glyceryl monolaurate and polyethylene-polypropylene glycol.

A tenth aspect provides non-aqueous pharmaceutical composition of the ninth aspect, wherein the ratio of glyceryl monolaurate to polyethylene-polypropylene glycol is about 1:1.

A eleventh aspect provides the non-aqueous pharmaceutical composition of the ninth aspect, wherein the glyceryl monolaurate is IMWITOR 312.

A twelfth aspect provides the non-aqueous pharmaceutical composition of the ninth aspect, wherein the polyethylene-polypropylene glycol is POLOXAMER 188.

A thirteenth aspect provides the non-aqueous pharmaceutical composition of the fourth aspect, wherein the at least two non-aqueous pharmaceutically acceptable excipients are glyceryl caprylate and polyethylene-polypropylene glycol.

A fourteenth aspect provides the non-aqueous pharmaceutical composition of the thirteenth aspect, wherein the ratio of glyceryl caprylate to polyethylene-polypropylene glycol is about 2:1.

A fifteenth aspect provides the non-aqueous pharmaceutical composition of the thirteenth aspect, wherein the glyceryl caprylate is CAPMUL MCM.

A sixteenth aspect provides the non-aqueous pharmaceutical composition of the thirteenth aspect, wherein the polyethylene-polypropylene glycol is POLOXAMER 188.

A seventeenth aspect provides the non-aqueous pharmaceutical composition of the fourth aspect, wherein the at least two non-aqueous pharmaceutically acceptable excipients are a polyethylene glycol and a polyethylene glycol monostearate.

A eighteenth aspect provides the non-aqueous pharmaceutical composition of the seventeenth aspect, wherein the polyethylene glycol has a molecular weight of at least about 1000 g/mol.

An nineteenth aspect provides the non-aqueous pharmaceutical composition of the seventeenth or eighteenth aspect, wherein the ratio of the polyethylene glycol to the polyethylene glycol monostearate is about 1:1.

A twentieth aspect provides the non-aqueous pharmaceutical composition of the seventeenth aspect, wherein the polyethylene glycol monostearate is MYRJ 52.

A twenty-first aspect provides the non-aqueous pharmaceutical composition of the fourth aspect, wherein the at least two non-aqueous pharmaceutically acceptable excipients are glyceryl monolaurate and a polysorbate.

A twenty-second aspect provides the non-aqueous pharmaceutical composition of the twenty-first aspect, wherein the ratio of glyceryl monolaurate and polysorbate is about 4:1.

A twenty-third aspect provides the non-aqueous pharmaceutical composition of the twenty-first or twenty-second aspect, wherein the polysorbate is polysorbate 80.

A twenty-fourth aspect provides the non-aqueous pharmaceutical composition of the twenty-first aspect, wherein the glyceryl monolaurate is IMWITOR 312.

A twenty-fifth aspect provides the non-aqueous pharmaceutical composition of the fourth aspect, wherein the at least two non-aqueous pharmaceutically acceptable excipients are propylene glycol and a polyethylene-polypropylene glycol.

A twenty-sixth aspect provides the non-aqueous pharmaceutical composition of the twenty-fifth aspect, wherein the ration of propylene glycol to the polyethylene-polypropylene glycol is about 1:4.

A twenty-seventh aspect provides the non-aqueous pharmaceutical composition of the twenty-fifth or twenty-sixth aspect, wherein the polyethylene-polypropylene glycol is POLOXAMER 188.

A twenty-eight aspect provides the non-aqueous pharmaceutical composition of the twenty-sixth aspect, wherein the at least two non-aqueous pharmaceutically acceptable excipients are a polyethylene glycol and a polyethylene-polypropylene glycol.

A twenty-ninth aspect provides the non-aqueous pharmaceutical composition of the twenty-eighth aspect, wherein the polyethylene glycol has a molecular weight of at least about 1500 g/mol.

A thirtieth aspect provides the non-aqueous pharmaceutical composition of the twenty-eighth or twenty-ninth aspect, wherein the ratio of polyethylene glycol to polyethylene-polypropylene glycol is about 7:3.

A thirty-first aspect provides the non-aqueous pharmaceutical composition of the twenty-eighth aspect, wherein the polyethylene-polypropylene glycol is POLOXAMER 188.

A thirty-second aspect provides the non-aqueous pharmaceutical composition of the fourth aspect, wherein each of the pharmaceutically acceptable excipients has a molecular weight of at least 200 g/mol.

A thirty third aspect provides a method of treating chronic lymphocytic leukemia, Hodgkin's disease, non-Hodgkin's lymphoma, multiple myeloma or breast cancer, in a patient in need thereof, comprising administering to said patient a pharmaceutically effective amount of a pharmaceutical composition according to any one of the preceding aspects.

A thirty-fourth aspect provides the use of a non-aqueous pharmaceutical composition of any one of the first through thirty-second aspects, for the manufacture of a medicament for the treatment of chronic lymphocytic leukemia, Hodgkin's disease, non-Hodgkin's lymphoma, multiple myeloma or breast cancer.

A thirty-fifth aspect provides the use of the thirty-fourth aspect, wherein the non-Hodgkin's lymphoma is indolent B-cell non-Hodgkin's lymphoma

A thirty-sixth aspect provdes a non-aqueous oral dosage form comprising the non-aqueous pharmaceutical composition of any one of the first through thirty-second aspects.

A thirty seventh aspect provides the non-aqueous oral dosage form of the thirty-fifth aspect, wherein the dosage form is a capsule, soft gel, immediate-release tablet, controlled-release tablet, extended-release tablet, or sachet.

What is claimed is:

1. A non-aqueous pharmaceutical composition for oral administration comprising:
bendamustine, or a pharmaceutically acceptable salt thereof; and
at least one non-aqueous pharmaceutically acceptable excipient selected from the group consisting of solvents and co-solvents, surfactants and co-surfactants, medium chain monoglycerides, and triglycerides.

2. A non-aqueous pharmaceutical composition for oral administration comprising:
bendamustine, or a pharmaceutically acceptable salt thereof; and
at least two non-aqueous pharmaceutically acceptable excipients selected from the group consisting of solvents and co-solvents, surfactants and co-surfactants, medium chain monoglycerides, and triglycerides.

3. The non-aqueous pharmaceutical composition of claim 1 wherein the at least one non-aqueous pharmaceutically acceptable excipient is selected from the group consisting of propylene carbonate, propylene glycol, glyceryl caprylate, polysorbates, polyethylene-polypropylene glycols, corn oil, glyceryl monolaurates, polyethylene glycol monostearates, polyethylene glycol monolaurates, polyethylene glycol dilaurates, polyethylene glycol hydroxyl stearates, triglycerides, polyethylene glycol distearates, polyethylene glycol tocopherols, and polyethylene glycols.

4. The non-aqueous pharmaceutical composition of claim 2 wherein the at least two non-aqueous pharmaceutically acceptable excipients are selected from the group consisting of propylene carbonate, propylene glycol, glyceryl caprylate, polysorbates, polyethylene-polypropylene glycols, corn oil, glyceryl monolaurates, polyethylene glycol monostearates, polyethylene glycol monolaurates, polyethylene glycol dilaurates, polyethylene glycol hydroxyl stearates, triglycerides, polyethylene glycol distearates, polyethylene glycol tocopherols, and polyethylene glycols.

5. A non-aqueous pharmaceutical composition for oral administration comprising:
bendamustine, or a pharmaceutically acceptable salt thereof; and
at least one non-aqueous pharmaceutically acceptable excipient selected from the group consisting of a polyethylene glycol monostearate, a polyethylene-polypropylene glycol, tocopherol polyethylene glycol 1000 succinate, a polyethylene glycol, a polyethylene glycol mono- and dilaurate mixture, a glyceryl laurate, and a polyethylene glycol hydroxystearate mixture.

6. A non-aqueous pharmaceutical composition according to claim 5 wherein the at least one non-aqueous pharmaceutically acceptable excipient is selected from the group consisting of MYRJ 52, POLOXAMER 188, SPEZIOL TPGS, PEG 1450, GELUCIRE 44/14, IMWITOR 312, and SOLUTOL HS15.

7. The non-aqueous pharmaceutical composition of claim 2 or 4, wherein the pharmaceutical composition is a solid solution, solid suspension, solid dispersion, liquid dispersion, suspension, emulsion, microemulsion, gel, or solution.

8. The non-aqueous pharmaceutical composition of claim 1 or 3, wherein the pharmaceutical composition is a solid solution, solid suspension, solid dispersion, liquid dispersion, suspension, gel, or solution.

9. The non-aqueous pharmaceutical composition of claim 4, wherein the at least two non-aqueous pharmaceutically acceptable excipients are glyceryl monolaurate and polyethylene-polypropylene glycol.

10. The non-aqueous pharmaceutical composition of claim 9, wherein the ratio of glyceryl monolaurate to polyethylene-polypropylene glycol is about 1:1.

11. The non-aqueous pharmaceutical composition of claim 9, wherein the glyceryl monolaurate is IMWITOR 312.

12. The non-aqueous pharmaceutical composition of claim 9, wherein the polyethylene-polypropylene glycol is POLOXAMER 188.

13. The non-aqueous pharmaceutical composition of claim 4, wherein the at least two non-aqueous pharmaceutically acceptable excipients are glyceryl caprylate and polyethylene-polypropylene glycol.

14. The non-aqueous pharmaceutical composition of claim 13, wherein the ratio of glyceryl caprylate to polyethylene-polypropylene glycol is about 2:1.

15. The non-aqueous pharmaceutical composition of claim 13, wherein the glyceryl caprylate is CAPMUL MCM.

16. The non-aqueous pharmaceutical composition of claim 13, wherein the polyethylene-polypropylene glycol is POLOXAMER 188.

17. The non-aqueous pharmaceutical composition of claim 4, wherein the at least two non-aqueous pharmaceutically acceptable excipients are a polyethylene glycol and a polyethylene glycol monostearate.

18. The non-aqueous pharmaceutical composition of claim 17, wherein the polyethylene glycol has a molecular weight of at least about 1000 g/mol.

19. The non-aqueous pharmaceutical composition of claim 17 or claim 18 wherein the ratio of the polyethylene glycol to the polyethylene glycol monostearate is about 1:1.

20. The non-aqueous pharmaceutical composition of claim 17, wherein the polyethylene glycol monostearate is MYRJ 52.

21. The non-aqueous pharmaceutical composition of claim 4, wherein the at least two non-aqueous pharmaceutically acceptable excipients are glyceryl monolaurate and a polysorbate.

22. The non-aqueous pharmaceutical composition of claim 21, wherein the ratio of glyceryl monolaurate and polysorbate is about 4:1.

23. The non-aqueous pharmaceutical composition of claim 21 or claim 22, wherein the polysorbate is polysorbate 80.

24. The non-aqueous pharmaceutical composition of claim 21, wherein the glyceryl monolaurate is IMWITOR 312.

25. The non-aqueous pharmaceutical composition of claim 4, wherein the at least two non-aqueous pharmaceutically acceptable excipients are propylene glycol and a polyethylene-polypropylene glycol.

26. The non-aqueous pharmaceutical composition of claim 25, wherein the ration of propylene glycol to the polyethylene-polypropylene glycol is about 1:4.

27. The non-aqueous pharmaceutical composition of claim 25 or 26, wherein the polyethylene-polypropylene glycol is POLOXAMER 188.

28. The non-aqueous pharmaceutical composition of claim 26, wherein the at least two non-aqueous pharmaceutically acceptable excipients are a polyethylene glycol and a polyethylene-polypropylene glycol.

29. The non-aqueous pharmaceutical composition of claim 28, wherein the polyethylene glycol has a molecular weight of at least about 1500 g/mol.

30. The non-aqueous pharmaceutical composition of claim 28 or claim 29, wherein the ratio of polyethylene glycol to polyethylene-polypropylene glycol is about 7:3.

31. The non-aqueous pharmaceutical composition of claim 28, wherein the polyethylene-polypropylene glycol is POLOXAMER 188.

32. The non-aqueous pharmaceutical composition of claim 4, wherein each of the pharmaceutically acceptable excipients has a molecular weight of at least 200 g/mol.

33. A method of treating chronic lymphocytic leukemia, Hodgkin's disease, non-Hodgkin's lymphoma, multiple myeloma or breast cancer, in a patient in need thereof, comprising administering to said patient a pharmaceutically effective amount of a pharmaceutical composition according to claim 1 or claim 2.

34. Use of a non-aqueous pharmaceutical composition according to claim 1 or claim 2, for the manufacture of a medicament for the treatment of chronic lymphocytic leukemia, Hodgkin's disease, non-Hodgkin's lymphoma, multiple myeloma or breast cancer.

35. The use of claim 34 wherein the non-Hodgkin's lymphoma is indolent B-cell non-Hodgkin's lymphoma

36. A non-aqueous oral dosage form comprising the non-aqueous pharmaceutical composition according to claim 1 or claim 2.

37. The non-aqueous oral dosage form of claim 36, wherein the dosage form is a capsule, soft gel, immediate-release tablet, controlled-release tablet, extended-release tablet, or sachet.
